(19) United States
(12) Patent Application Publication
(10) Pub. No.: US 2006/0241742 A1
(43) Pub. Date: Oct. 26, 2006

(54) Stent comprising a coating system

(75) Inventors: Claus Harder, Uttenreuth (DE); Stephane Delaloye, Buelach (CH); Bernd Heublein, Hannover (DE)

Correspondence Address: HAHN LOESER & PARKS, LLP, One GOJO Plaza, Suite 300, AKRON, OH, 44311-1076, US

(73) Assignee: BIOTRONIK GmbH & Co. KG, Berlin (DE)

(21) Appl. No.: 10/562,376

(22) PCT Filed: Jun. 14, 2004
(86) PCT No.: PCT/EP04/06379

§ 371 Date: May 2, 2006

(30) Foreign Application Priority Data
Jun. 23, 2003 (DE) 103 29 260.8

(51) Int. Cl. A61F 2/82

(52) U.S. Cl. 623/1.42

(57) ABSTRACT

A stent comprises a tubular base body which is open on the front sides thereof and has a peripheral wall that is at least partially covered with a coating system consisting of at least one polymer carrier and at least one pharmacologically active substance, which is released into the surrounding tissue once the stent has been implanted in the human or animal body. The invention creates a coating system which enables an optimum local application of the active ingredient. A concentration of the substance, a morphological structure of the carrier(s), a material modification of the carrier(s), and/or a layer thickness of the carrier(s), are predetermined in the longitudinal direction of the stent, in such a way that the elution varies locally in the longitudinal direction of the stent and is determined according to the pathophysiological and/or rheological conditions to be expected during the application.

### BACKGROUND OF THE INVENTION

The invention relates to stents with coating systems of one or more polymer carriers and at least one pharmacologically active substance, whereby after implantation of the stent the substance is released into the surrounding tissue in the human or animal body.

Coronary heart diseases, in particular myocardial infarctions, are one of the most frequent causes of death in Western Europe and North America. In more than 80% of cases the cause of the myocardial infarction is thrombolytic occlusion of the coronary artery through rupture of atheromatous plaque in pre-existing stenosing atheromatosis. Key factors for the long-term prognosis after an acute myocardial infarction are:
an effective and long-lasting reopening of the infarction arteries
duration of the thrombolytic vascular occlusion
prevention of major myocardial loss and ventricular remodelling
the controlling of rhythmogenic complications

The aforementioned factors do not only determine the cardiovascular mortality, but also the quality of life after the infarction.

For more than twenty years, non-surgical methods of treating stenoses have been established, in which, including through balloon dilation (PTCA Percutaneous Transluminal Coronary Angioplasty), the constricted or blocked blood vessel is dilated again. This procedure has particularly proven its worth in the treatment acute myocardial infarction. However, when dilating the blood vessel minute injuries, tears, dissections in the vascular wall occur, which although they often heal without problems, in around one third of cases lead to proliferation because of the triggered cell growth, which eventually results in renewed vascular constriction (restenosis). Dilation also does not eliminate the causes of stenosis, i.e. the physiological changes in the vascular wall. Another cause of restenosis is the elasticity of the dilated blood vessel. After removal of the balloon the blood vessel contracts excessively so that the cross-section of the blood vessel is reduced (obstruction, known as negative remodelling). The latter effect can only be prevented by the application of a stent.

In the surgical treatment of stable angina pectoris in coronary heart disease, the insertion of a stent has resulted in a considerable reduction in the rate of restenosis and thus to improved long-term results. This applies both to primary and relapse stenosis. The benefit of stent implantation is based on the greater primary lumen gain.

Although the use of stents can achieve an optimum vascular cross-section, the implantation of stents also leads to minute injuries which can induce proliferation and eventually trigger restenosis. Furthermore, the presence of a foreign body of this type initiates a cascade of microbiological processes which can lead to a gradual closing of the stent.

In the meantime, extensive knowledge about the cell-biological mechanism and the trigger factors for stenosis and restenosis has been gained. As has already been stated, restenosis occurs as a reaction of the vascular wall to local injury due to dilation of the atherosclerotic plaque. Via complex action mechanisms, the lumen-directed migration and proliferation of the smooth muscle cells of the media and adventitia is induced (neointimal hyperplasia). Under the effect of various growth factors the smooth muscle cells produce a coating layer of matrix proteins (elastin, collagen, proteoglycane), the uncontrolled growth of which can gradually lead to constriction of the lumen. Systemic drugs treatment includes the oral administration of calcium antagonists, ACE inhibitors, anticoagulants, anti-aggregants, fish oils, antiproliferative substances, anti-inflammatory substances and serotonin antagonists, but significant reductions in the types of restenosis have so far not been achieved.

For some years, attempts have been made to reduce the risk of restenosis during the implantation of stents by applying special coating systems. The coating systems partly act as carriers in which one or more pharmacologically active substances are embedded (Local Drug Delivery, LDD). As a rule, the coating layers cover at least one circumference wall of the endovascular implant facing the vascular wall. So far, numerous preparations have been proposed as active substances or combinations of active substances for LDD systems.

The carrier of such coating systems comprises a biocompatible material, which can be either of natural origin or obtained synthetically. Biodegradable coating materials provide particularly good compatibility and the opportunity of influencing the elution characteristics of the embedded medicinal product. Examples of the use of biodegradable polymers are cellulose, collagen, albumin, casein, polysaccharide (PSAC), polylactide (PLA), poly-L-lactide (PLLA), polyglycol (PGA), poly-D,L-lactide-co-glycolide (PDLLA/PGA), polyhydroxy butyric acid (PHB), polyhydroxyvaleric acid (PHV), polyalkylcarbonate, polyorthoester, polyethylene terephthalate (PET), polymalonic acid (PML), polyanhydrides, polyphosphazenes, polyamino acids and their copolymers, as well as hyaluronic acid and its derivatives.

To apply the coating systems to the stent, several methods have been developed, such as rotation atomization methods, immersion methods and spraying methods. The coating system covers at least parts of the circumferential wall of the stent facing the vascular wall. In the human or animal body, release of the pharmacologically active substances takes place through gradual degradation of the carrier and/or diffusion into the surrounding tissue. The elution characteristics of the substances can be assessed in advance using established in vitro tests.

Known LDD stents exhibit no locally differentiated elution characteristics for the substances. Thus, the coating systems in the area of the open surfaces of the tubular of basic body of the stent as well as in the middle area of the stent are of approximately the same quality. However, particularly in the case of long stenoses in which the nature of the lesion changes over the length of the stent, such costing systems can be disadvantageous. This can apply, for example, in the case of an elongated lesion to be treated with a particular substance, which has a very large quantity of plaque in the centre, which then decreases towards the outside. In homogenous substance treatment, the edge areas of the stent are overdosed in certain circumstances, which can promote proliferation in these areas, whereas the same dose in the middle area of the lesion has an anti-proliferative effect. Furthermore a discharge of the substances of and LDD stent at its ends, i.e. in the area of its open surfaces is increased which can lead to local underdosing.

### BRIEF SUMMARY OF THE INVENTION

On the basis of the state of the art an aspect is thus to create a coating system with which optimum local active substance application over the entire length of the stent becomes possible.

This aspect is achieved by way of the stent with the characteristics set out herein.

The invention is based on a stent with a tubular basic body open at its face surfaces, the circumferential wall of which is at least partially covered with a coating system of one or more polymer carriers and at least one pharmacologically active substance. The stent in accordance with the invention is characterised in that one or more parameters of the coating system, namely
a concentration of the substance
a morphological structure of the carrier(s)
a material modification of the carrier(s) and/or
a coating thickness of the carrier(s)
   is/are predetermined in the longitudinal direction of the stent so that the substance exhibits predetermined locally different elution characteristics in the longitudinal direction of the stent depending on the pathophysiological and/rheological conditions to be expected of the application. In this way it is possible to release to differing degrees the at least one substance over the length of the stent into the adjacent tissue.

The term “coating system” in the sense of the invention is taken to mean the combination of a polymer, possibly biodegradable carrier, with at least one pharmacologically active substance. The coating system covers at least some areas of an outer surface of the stent.

“Pharmacologically active substance” is taken to mean a medicinal product which in a suitable dose acts as a therapeutic agent to influence physical conditions or functions as a substitute for natural active substances produced by the human or animal body as well as to eliminate or render harmless disease pathogens or exogenous substances.

“Local elution characteristics” is taken to mean the release of a substance into the adjacent tissue environment over a certain period of time, limited in spatial terms to a predefined partial area of the coated stent.

For example, if the concentration of the substance in a middle section of the stent is increased, the local dose in this section is also increased. If a local lesion extends in the section of the stent, it can be treated in a highly potent manner with an optimal dose. In the direction of the face surface, the dosage of the substance decreases so that the promotion of proliferation is prevented.

On the other hand there is a tendency for neointima formation at the ends of the stent. It is therefore sensible to establish coating systems in these areas which have a neointima formation inhibiting or suppressing substance in concentrations higher than in the middle sections of the stent. In this way the dose of the substance is increased in the vascular tissue facing the ends.

By varying the layer thickness of the carrier the local elution characteristics can be influenced over the length of the stent. Here maintaining the dose over a certain period of time is at the forefront. However, depending on the pathophysiological circumstances in the individual sections of the vessels facing the circumferential wall of the stent, it is necessary to maintain the medicinal treatment over a certain period of time. With an increased layer thickness the dosage period can be increased. Depending on the application, the morphological structure, material modification as well the concentration of the substance can of course be varied.

“Morphological structures” in accordance with the invention is taken to mean the conformation and aggregation of the polymers forming the carriers. This includes the type of molecular order structure, the porosity, the surface quality and other intrinsic properties of the carrier that influence diffusion of the active substance or its degradation behaviour. Molecular order structures cover amorphic (partial) crystalline or mesomorphic polymer phases that can be influenced and/or generated in dependence on the used production process, coating process and environmental conditions. Through specific variation of the production and coating process the porosity and surface quality of the carrier can be influenced. In general with increasing porosity of the carrier the more quickly the active substance is released. Amorphic structures have similar effects vis-a-vis (partial) crystalline structures.

“Material modification” is here taken to mean the blend production of polymers as well as the addition of fillers and additives in order to influence the elution characteristics.

Preferably, the carrier is made of a biodegradable polymer so that after implantation of the stent in the human or animal body the substance is also released through gradual degradation of the carrier into the surrounding tissue. The degradation behaviour of the carrier thus constitutes a further parameter with which active substance release can be controlled, i.e. with which a differentiation of the elution characteristics in accordance with the invention is possible. More rapid degradation of the carrier leads to quicker release of the substance. The degradation rate of the biodegradable polymer is not only dependent on the polymer carrier material present, but can also be influenced by variation of the morphological structure and through material modifications.

In accordance with the invention, the local elution characteristics of the substance are in the axial direction, i.e. over the length of the stent, predetermined depending on the pathophysiological and/or rheological conditions anticipated in the application. The pathophysiological aspects take into account the fact that as a rule the stent is placed in the vessel in such a way that it is positioned centrally on the lesion, i.e. the tissue adjacent to the ends and the middle section of the stent is of a different nature. Rheological aspects, in turn, take into account the fact that the flow conditions, particularly in the area of the ends and in the middle sections of the stent are different. Thus at the ends of the stent there may be greater release of the substance due to a stronger flow. Rheological parameters can vary strongly depending on the design of the stent and must be determined in individual cases. By taking the two above parameters into consideration for LDD treatment optimum dosage can be ensured over the entire stent dimensions.

Preferably, the release behaviour of different polymer carriers is also included. If one or more of the carriers are biodegradable, in order to vary the local elution characteristics the degradation behaviour of the carrier(s) can be influenced in the manner described above. For example, in order to increase the local dosage, a more rapidly degrading carrier in a particular stent area can be envisaged than in other available stents. More rapid degradation of the stent in this area leads to a local increase in the dosage of the substance which, as such, is also present in the other carriers in the same concentrations.

Such a system can be used, for example, if an increase in the concentration of the substance in the carrier material leads to undesirable crystallisation processes, which in turn negatively influence the release behaviour and long-term stability.

The coating system in accordance with the invention can be described referring back to conventional coating techniques. For application purposes, conventional masking methods can be used.

### BRIEF SUMMARY OF THE SEVERAL VIEWS OF THE DRAWINGS

The invention will be described below in more detail with the aid of examples of embodiment and the accompanying drawings.

FIG. 1 shows a stent with a tubular basic body open at its face surfaces, the circumferential wall of which is covered with a coating system;

FIGS. 2a, 2b show a schematic cross-section along a longitudinal axis of a stent to illustrate a first variant of the coating system in accordance with the invention;

FIGS. 3a, 3b show a schematic cross-section along a longitudinal axis of a stent to illustrate a second variant of the coating system in accordance with the invention;

FIG. 4 shows a schematic cross-section along a longitudinal axis of a stent to illustrate a third variant of the coating system in accordance with the invention;

FIG. 5 shows a schematic cross-section along a longitudinal axis of a stent to illustrate a fourth variant of the coating system in accordance with the invention.

### DETAILED DESCRIPTION OF THE INVENTION

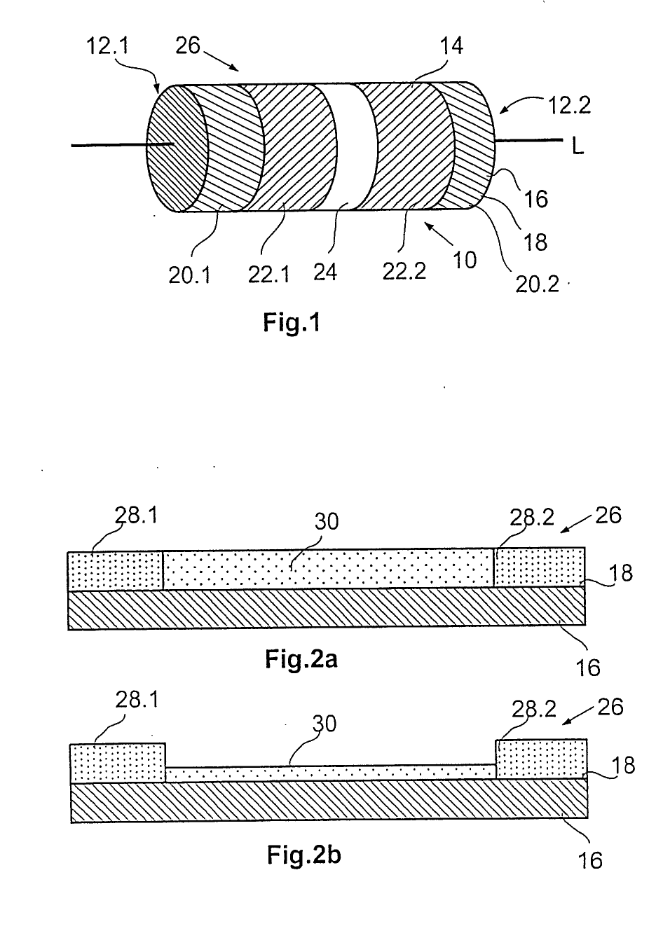

FIG. 1 shows a strongly schematic perspective side view of a stent 10 with a tubular basic body 14 open at its ends 12.1, 12.2. A circumferential wall 16 of the basic body 14 extending radially about a longitudinal axis L comprises segments arranged next to each other in the axial direction which in turn are composed of a number of support elements arranged in a particular pattern. The individual segments are connected to each other by means of connection links together resulting in the basic body 14. In FIG. 1, the illustration of a specific stent design was consciously avoided as this is not necessary to show the coating system in accordance with the invention and also because for each stent design individual adaptation to the relevant geometric factors and other parameters is necessary. Large numbers of the most varied stent designs are known from the state of the art and are not therefore described in more detail here. All that has to be emphasised is that all current stents 10 have a basic framework of any shape which has a surrounding circumferential wall 16. In the following, an external surface sheath 18 of the circumferential wall 16 is equated with the outer circumferential surface possibly formed of a multiplicity of present support elements.

The stent 10 in FIG. 1 shows in a strongly schematic manner a coating system 26 in which several sections 20.1, 20.2, 22.1, 22.2, 24 of the external surface sheath 18 of the circumferential wall 16 are provided with coatings with diverging properties.

The differences in the coatings in the individual sections 20.1, 20.1, 202, 22.1, 22.2, 24 consist in the fact that the individual coating sections comprising biodegradable carriers and pharmacologically active substance differ in their local elution characteristics for the pharmacologically active substance. Thus, as will be described in more detail, it can be envisaged that the sections 20.1 and 20.2 at the ends 12.1, 12.2 of the stent release the substance over time at a first dose, which for this substance is higher than in sections more strongly arranged in the middle 22.1, 22.2 and 24. This in turn means that after implantation the tissue areas of the vascular wall facing sections 20.1, 20.2, 22.1, 22.2 and 24 are exposed to different doses of the substance. In each case the coating system therefore has two or more sections with locally different elution characteristics for the substance.

FIGS. 2a, 2b, 3a, 3b, 4 and 5 each show strongly schematically, a cross-section along the longitudinal axis L of the stent 10, and in each case only the two resulting sections through the circumferential wall 16. However, beforehand the fundamental principles of designing the individual coating systems are briefly explained.

The local elution characteristics of one or more substances present in the coating system essentially depend on five factors:
a) a concentration of the substances in the carrier(s)
b) a layer thickness of the carrier,
c) a degradation behaviour of the carrier,
d) a morphological structure of the carrier and
e) a material modification of the structure.

Point a) takes into account the fundamental principal that increasing the concentration of the active substance is associated with a higher dose. However, this phenomenon does not necessarily have to be linear and both the dose and the release duration are influenced by further factors. The principle of active substance release through diffusion has, however, been undermined both theoretically and practically be numerous examples, so that on the one hand theoretical statements are possible regarding in vivo release and on the other hand in vitro experiments can simulate processes actually occurring in the body with a high degree of accuracy.

A variation in the layer thickness of the carrier (point b)) with an unchanged concentration of embedded substance generally influences the dosage duration. However, other effects can occur in the phase interfaces which also have an effect on the release of the substance and thus on the dose of the substance over a particular period of time. Here too, there are well-founded theoretical and practical model systems, which allow assessment of subsequent in vivo behaviour.

Another factor influencing the local elution characteristics is the degradation behaviour of the biodegradable carrier (point c)). With the gradual breakdown of the carrier the substance embedded in these areas is released. Generally, two diffusion processes take place in parallel. Depending on the solubility of the substance, it is quite possible for the degradation of the carrier to take place much more rapidly that the gradual dissolution of the substance. Thus, under certain circumstances the substances can be absorbed by the surrounding tissue in the form of microparticles or nanoparticles. Sound scientific knowledge about the degradation behaviour of individual carrier systems is already available. On the basis of this and in vitro experiments running in parallel, the behaviour of equivalent carrier systems can be predicted in the living organism.

Finally, the local elution characteristics depend on the morphological structure and material modifications of the carriers (points d) and e)). Thus, the porosity of the carriers can differ in particular, whereby greater porosity leads to accelerated degradation and increased diffusion. With regard to material modification the mixing of additives to the carriers can be envisaged which delay enzymatic breakdown.

In summary, it can therefore be stated that depending on the variability of the system, i.e. whether, for example, several carrier systems are present, or the concentrations of the one or more substances change, or the layer thicknesses of the carriers are changed, the elution characteristics of more or more substances can be adjusted.

Adjustment of the individual sections of the coating system of the stent is therefore carried out in dependence on the pathophysiological and rheological conditions to be expected of the application.

The pathophysiological conditions are here taken to mean the tissue structure in the entire vascular area that has been altered by disease. Generally, the stent is positioned in such a way that the lesion, i.e. in coronary applications usually the fibroatheromatous plaque, is in the middle area of the stent. In other words, the adjacent tissue structures diverge axially over the length of the stent whereby in certain circumstances other treatment is locally indicated.

The rheological conditions are taken to mean the flow conditions brought about in individual longitudinal sections of the stent after implantation of the stent. Experience has shown that the flow around the ends of the stent is stronger than in the middle sections of the stent. This can result in degradation of the carrier or diffusion of the substance being increased in the end areas.

In every conventional drugs therapy, optimum doses are aimed for at the site of action in order to support the healing process. However, this must also apply at local level if the tissue structures in this local area require different treatment. Thus, too small a dose cannot support the healing process and too high a dose can, counterproductively, trigger inflammatory processes.

All polymer matrices of a synthetic nature or of natural origin that can be broken down in the living organism through enzymatic or hydrolytic processes can be used as biodegradable carriers in accordance with the invention. In particular, polymers from the group cellulose, collagen, albumin, casein, polysaccharide (PSAC), polylactide (PLA), poly-L-lactide (PLLA), polyglycol (PGA), poly-D,L-lactide-co-glycolide (PDLLA/PGA), polyhydroxy butyric acid (PHB), polyhydroxyvaleric acid (PHV), polyalkylcarbonate, polyorthoester, polyethylene terephthalate (PET), polymalonic acid (PML), polyanhydrides, polyphosphazenes, polyamino acids and their copolymers, as well as hyaluronic acid can be used. Depending on the desired characteristics of the coating system the polymers can be applied in pure form, in derivative form, in the form of blends or as copolymers.

As pharmacologically active substances used in particular to treat the effects of percutaneous coronary interventions, calcium antagonists, ACE inhibitors, anticoagulants, anti-aggregants, fish oils, antiproliferative substances, immunosuppressants, chemotherapeutic agents, anti-inflammatory substances, serotonin antagonits as well as PPAR and RXR agonists are suitable for example.

FIG. 2a shows a strongly schematic and simplified section of the circumferential wall 16 with its coating system 26 applied to the external sheath area 18. The coating system 26 comprises two end sections 28.1 and 28.2 as well as a middle section 30. In this case the entire coating system 26 is formed of a biodegradable carrier and pharmacologically active substance applied in an even layer thickness.

Sections 28.1 and 28.2, 30 differ in that the pharmacologically active substance is embedded in the carrier higher and lower concentrations. Thus, in this case the concentration of the substance in the end sections 28.1, 28.2 is increased compared with the middle section 30. Optionally, the transition from a low concentration to a higher concentration can also be continuous over the entire length of the stent.

The coating system 26 shown in FIG. 1 is particularly suitable for two case constellations. On the one hand, in rheological conditions bringing about increased discharge of the substance in the end areas largely even dosing over the entire stent length can be assured. On the other hand, it is possible to apply an increased dose in the end areas so that the pathophysiological tissue differences over the entire length of the stent are looked into in more detail. In this way, the neointima formation inhibiting substances in particular can be made available in increased concentrations.

FIG. 2b discloses a second variant of a coating system 26 comprising a carrier and a pharmacologically active substance. The sections 28.1, 28.2 correspond to those in FIG. 2a. In contrast, the layer thickness of section 30 is considerably reduced. The result of this is that the dose of the pharmacologically active substance is reduced in the opposite tissue areas, i.e. more particularly, the dosage duration is shortened. Such a layer arrangement makes sense, for example, if the pharmacologically active substance is only to reach the lesion area for a short period of time after which there may be an undesirable effect on the healing process.

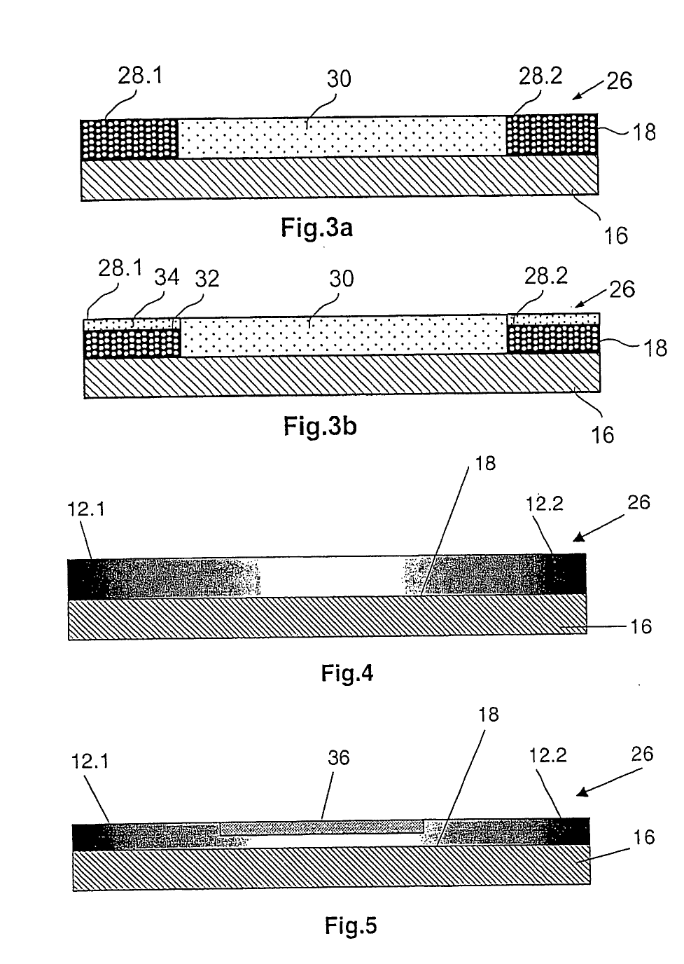

FIG. 3a shows a coating system 26 in which two different carriers with different degradation behaviours are applied in sections 28.1, 28.2, 30 of the stent 10. The same applies to the variation of the system in accordance with FIG. 3b. In both coating systems 26 only one substance is distributed in a homogenous concentration over both carriers.

In accordance with the embodiment in FIG. 3a, the sections 28.1, 28.2 are covered with a carrier with delayed degradation behaviour compared with the carrier used in the middle section 30. Accordingly the local elution characteristics of the substance are influenced, i.e. generally delayed at the ends. Such an embodiment is always useful if the dose at the ends is to be maintained over a longer period of time or, on the basis of the rheological conditions discharge of the substances is to be counteracted.

In sections 28.1 and 28.2 FIG. 3b exhibits a multiple layer structure of the coating system 26 in the radial direction. In a first partial section 32, the carrier is again applied with the delayed degradation behaviour, whereas radially outwards there is a partial section 34 with the more rapidly degrading carrier.

FIG. 4 shows a coating system 26 in which two different pharmacologically active substances are applied to one single carrier. A concentration of the substance changes axially over the length of the stent in a continuous manner. In order to clarify the course of the concentration of both substances a schematic depiction of the course was chosen. A concentration of a first substance is shown by way of progressing darkness and that of a second substance by way of progressing lightness. Thus a concentration of the first substance is greatly increased at the ends 12.1, 12.2 of the stent, whereas its concentration reduces sharply in the middle section. Inversely, the second substance is present in increased concentration in the middle sections of the coating system 26 and decreases towards the ends 12.1 and 12.2. Such a system is suitable, for example, for carrying out locally differentiated drugs treatment with the aid of the first substance at the ends 12.1 and 12.2 of the stent, and essentially treating the lesion with the second substance in the middle section of the stent.

In FIG. 5, the coating system 26 in FIG. 4 has been further differentiated in that an additional middle partial section 36 made of a different carrier, which also contains a further substance has been integrated into the coating system. The carrier of the partial section 36 exhibits a very rapid degradation behaviour and accordingly releases the further substance embedded in it very rapidly and at a higher dose. The first and second substances are subsequently released as has already been described in FIG. 4.

The above examples in FIGS. 2a, 2b, 3a, 3b, 4 and 5 only show strongly schematic examples of embodiment of the coating system 26 in accordance with the invention. They can be combined in a multitude of different ways. For example, it is conceivable to design a complex coating system consisting of several carrier systems each with different substances in individual sections. The primary aim is always to optimise the local dose of the substances in the opposing tissue sections.

We claim:

1. Stent A stent comprising a tubular basic body open at its face surfaces, the circumferential wall of which is covered at least in places with a coating system comprising one or more polymer carriers and at least one pharmaceutically active substance, whereby the substance, after implantation of the stent into a human or animal body, is released into the surrounding tissue, wherein one or more parameters of the coating system, selected from
a concentration of the substance
a morphological structure of the carrier(s)
a material modification of the carrier(s) and/or and
a layer thickness of the carrier(s)
is/are predetermined in the longitudinal direction of the stent so that the substance exhibits predetermined locally different elution characteristics in the longitudinal direction of the stent depending on the pathophysiological and/or Theological conditions to be expected of an application.

2. Stent The stent according to claim 1, wherein the polymer carrier is biodegradable.

3. The stent according to claim 2, wherein a degradation behaviour of the carrier serves to differentiate the local elution characteristics.
